(19) United States
(12) Patent Application Publication
(10) Pub. No.: US 2009/0090650 A1
(43) Pub. Date: Apr. 9, 2009

(54) METHOD FOR CATHETER LUBRICATION AND KIT THEREFOR

(76) Inventor: Ronald James Wells, Olympia, WA (US)

Correspondence Address: Oliver Law Firm, PS Inc., 7907 Alma Blvd., Waxhaw, NC, 28173, US

(21) Appl. No.: 12/243,458

(22) Filed: Oct. 1, 2008

Related U.S. Application Data
(60) Provisional application No. 60/977,296, filed on Oct. 3, 2007.

(51) Int. Cl. A61B 19/02; A61M 25/01

(52) U.S. Cl. 206/571; 604/544

(57) ABSTRACT

A completely new kit and method includes a device (connector strip with slotted hole) to hold steady a catheter for lubrication and insertion. This kit and method are designed to be used by patients who use an Intermittent Catheter technique. The connector strip comprises one portion of a two-part fastener, and the mating portion can be mounted at appropriate mounting location(s), such as the user's waistband(s). A small pouch, which can fit in a man's pants pocket, can stow all of the necessary elements of this kit.

### RELATED APPLICATIONS

This application is related to and claims priority under 35 U.S.C. 119(e) to U.S. provisional application Ser. No. 60/977,296, entitled “Medical Procedure,” filed on Oct. 3, 2007, with inventor Ronald James Wells, which is hereby incorporated by reference in its entirety.

### FIELD OF THE INVENTION

This invention pertains generally to medical procedures and more particularly to an improved method and kit for lubricating a catheter to be used in urology departments of hospitals and by urology patients while in their own homes or when away from home.

### BACKGROUND OF THE FIELD

Currently, many patients needing catheterization are limited to two options—“Turp” surgery or Self-catheterization. Surgery is often eschewed due to its inherent risks, leaving self-catheterization—or Intermittent Catheterization—as the remaining option. Self-catheterization can be uncomfortable, cumbersome, and messy. Users of the currently available self-catheterization methods may feel trapped in a difficult procedure, such that they do not have the freedom to travel or enjoy a full social life.

### SUMMARY OF THE INVENTION

The present invention solves the above-mentioned problems by providing a simple and effective way for a patient to hold steady a catheter for lubrication, to facilitate insertion, usage, and removal. The present invention may also include a kit for such lubrication, for traveling, etc.

One embodiment of the invention comprises a kit including a connector strip with a fastener mounted thereon, such connector strip being capable of temporarily mounting to a mounting location and of holding steady a catheter (typically with a hole for threading the catheter therethrough) for lubrication. The kit may also include a travel-sized bottle of lubricant, a plurality of cleansing towelettes, and/or other convenience items.

The fastener may be chosen from the two-part type—such as a hook-and-loop fastener—so that one portion may be mounted on the connector strip and the mating portion mounted on the mounting location(s). The user can then temporarily mount the connector strip to the mounting location(s) when needed. In a preferred embodiment, the mounting location is chosen to be on the user's clothing so that the user will always have a suitable mounting location with him. In this case, an optimum mounting location may be on the inside of the waistband of the user's pants, providing that the mounting location will be in proximity to the insertion point of the catheter.

In alternate embodiments, the mounting locations may be chosen as any suitable locations in the bathrooms or other locations such as portable stands or walkers. In this case, the user can use the most convenient mounting location available at the necessary time.

### BRIEF DESCRIPTION OF THE DRAWINGS

The objects, features, and advantages of the present invention will be apparent to one skilled in the art from- reading the following description in which:

FIG. 1 is a perspective view of a kit of a preferred embodiment;

FIG. 2 is a detail view of a connector strip with fastener portion mounted;

FIG. 3 is a detail view of a linking step threading the catheter into the slotted hole of the connector strip;

FIG. 4 is a detail view of a preferred mounting location for a fastener portion;

FIG. 5 is a detail view showing the threaded catheter;

FIG. 6 is a detail view showing the threaded catheter being held up and ready for lubrication;

FIG. 7 is a detail view showing an alternate mounting location for a fastener portion; and

FIG. 8 is a detail view showing a bottle being secured to the user's hand using a rubber band.

### DETAILED DESCRIPTION

The following specification describes apparati and method for improved catheter lubrication and kit therefor. In the description, specific materials and configurations are set forth in order to provide a more complete understanding of the present invention. But it is understood by those skilled in the art that the present invention can be practiced without those specific details. In some instances, well-known elements are not described precisely so as not to obscure the invention.

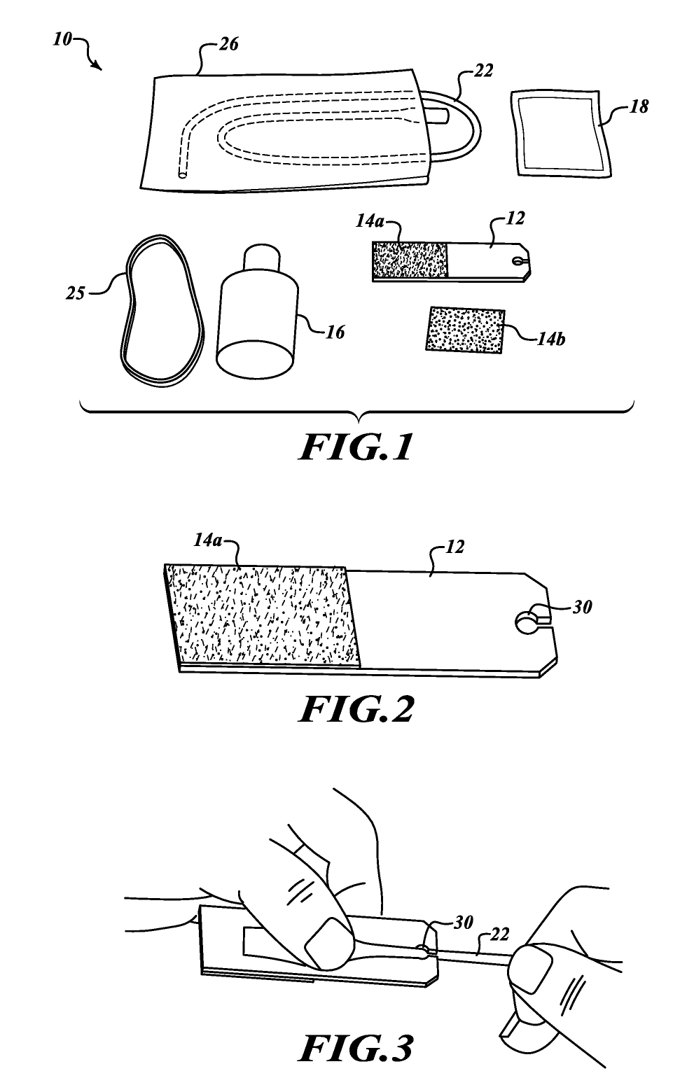

FIG. 1 shows the kit 10 of the preferred embodiment, including the basic elements of a plastic connector strip 12, a two-part hook-and-loop fastener with hook portion 14a mounted on the connector strip 12 and loop portion(s) 14b available for affixing to the mounting location(s), a plastic bottle 16, and a pouch 26 for carrying the elements of the kit. The kit may also include a plurality of cleansing towelettes 18, a zippered (or otherwise sealable) plastic bag (not shown) for stowing the catheter 22, an application brush (not shown) for applying glue to the loop portions 14b of the hook-and-loop fastener(s) to affix them to the chosen mounting locations, a rubber band 25, and instructions for use. The elements shown will usually fit easily within a man's pants pocket and can be carried inconspicuously to be used as necessary. It is noted that a catheter 22 typically requires a prescription and so is not a part of this invention, but can be carried in the pouch 26 with the rest of the kit 10 (the kit may include a plastic bag—such as a see-through Ziploc™ bag—for carrying the catheter). It is assumed that the user will already have the necessary catheter.

The particular lubricant used must also be provided by the user. However, an appropriately sized plastic bottle 16 is provided as part of the kit—usually what is considered a ‘travel size.’ The kit's bottle 16 may be filled from the user's larger lubricant bottle and is sized to hold a reasonable amount of lubricant for the kit's purpose. The inventor has found that an optimum size and type of bottle 16 may be an oval shape that holds approximately ¾ oz of lubricant. The bottle 16 is also sized to fit in the user's hand. (The kit's bottle 16 can be held in the user's hand using the rubber band 25 when countertops or shelves are not available, and this sort of information may be included in the instruction sheet.)

FIG. 2 shows details of the connector strip 12 of the preferred embodiment. The connector strip 12 is typically a generally flat, generally rectangular rigid piece of plastic (but could be wood, metal, or the like) with a slotted hole 30 at one end—located so that the hole is proximate an edge and the slot communicates the hole to the edge. (The invention could also be practiced with a round hole instead of a slotted hole, but the slot makes insertion of the catheter cleaner and easier.) The corners of the connector strip may be clipped (as shown), smoothed, rounded, or the like for safety and convenience. A first portion of the two-part, removable fastener will be permanently mounted at the opposing end—typically spaced apart from the slotted hole 30. Although any type of appropriate fastener could be used—such as snaps, hook-and-eye, or other type fasteners—in the preferred embodiment, hook-and-loop fasteners, such as Velcro™, are used, and the hook portion 14a is permanently mounted to the connector strip 12. This allows that the second mating portion(s), here the loop portion(s) 14b, the softer portions, can be affixed to the chosen mounting location(s) 32. In a preferred method, a chosen mounting location 32 for the second mating (loop) portion 14b is the inside waistband of the user's clothing (as shown in later figures). Steps for affixing the second loop portion(s) to the mounting location(s)—perhaps using an applicator such as an application brush that may be supplied in the kit—may be included in the instruction sheet.

FIG. 3 shows one way to thread the catheter 22 through the slotted hole 30. In the preferred embodiment of the connector strip 12, the slotted hole is sized to accommodate a catheter up to size #16. Alternate embodiments could be sized to accommodate different sizes of catheters. The user can stretch the catheter so as to insert it through the slot to be seated in the hole 30. A typical catheter has a flared end and a looped end. If the catheter also defines a flange, then the user can manipulate the catheter so that the flange rests against the connector strip. Once seated thusly, the catheter 22 will be held within the slotted hole 30, and the user can then removably attach the connector strip 12 to the mounting location 32 so that the flared end of the catheter points downward into the toilet. Again, these steps may be included in the instruction sheet.

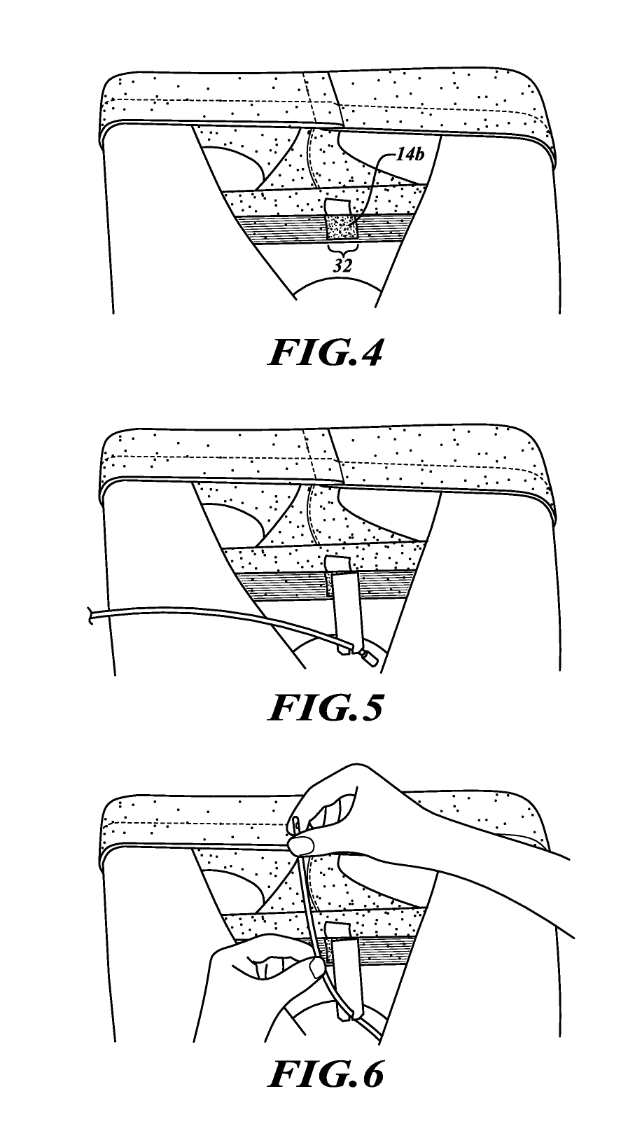

FIG. 4 shows a preferred choice for a mounting location 32. In the preferred embodiment shown, the loop portion 14b of the Velcro™ hook-and-loop fastener is permanently affixed to the inside waistband of the user's shorts/pants. This provides for a proximate mounting location for the connector strip 12 holding the catheter 22. If the user positions the mounting location 32 on the hard surface of the toilet seat, he can ensure a more stable base for the connector strip and the catheter 22. The mounting location shown is on the inside rear part of the user's waistband, a preferred location for use with the user in a seated position. However, the method may be used with the user in a standing position, in which case, the preferred mounting location 32 is on the inside front of the waistband.

Once the user has threaded the catheter 22 into the slotted hole 30 and mounted the connector strip to the mounting location 32 (as shown in FIG. 5) by temporarily mating the two portions 14a and 14b of the removable fastener, the catheter 22 may be lubricated as necessary before insertion and use. FIG. 6 shows the user holding up the catheter for lubrication. After subsequent removal of the catheter, the user cleans up and repacks his pouch 26 with the elements of the kit.

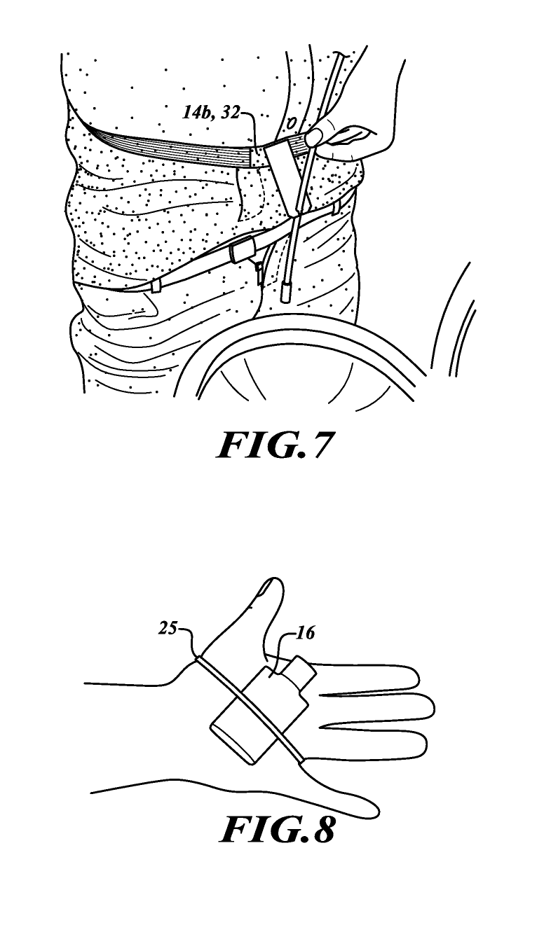

FIG. 7 shows an alternate mounting location 32 for the connector strip 12. In the case shown, the user has affixed a loop portion 14b of the hook-and-loop fastener on the inside of the front waistband of his shorts. This alternate mounting location 32 affords the user a convenient mounting location in a standing position, as opposed to sitting on the toilet.

FIG. 8 shows how the small lubricant bottle 16 can be securely held in the user's hand using the rubber band 25 to aid in the lubrication step. Strapping and securing the bottle 16 thusly can make the lubricant readily available when there are no countertops, shelves, or other horizontal surfaces available to the user for placing the bottle in proximity.

What is claimed is:

1. A kit for an Improved Method for Catheter Lubrication, such kit comprising: a connector strip, a bottle for lubricant, and a two-part fastener.

2. The kit of claim 1 wherein said connector strip is a generally flat, generally rectangular piece of plastic defining a hole at one end proximate an edge.

3. The kit of claim 2 wherein said hole is a slotted hole such that the hole communicates with said edge of said connector strip.

4. The kit of claim 2 wherein said two-part fastener has a first portion and a second portion, said first portion being permanently affixed to said connector strip and spaced apart from said hole.

5. The kit of claim 3 wherein said two-part fastener is a hook-and-loop fastener and said first portion is a hook portion and said second portion is a loop portion, said hook portion being permanently affixed to an opposing end of said connector strip from said slotted hole.

6. The kit of claim 1 wherein said bottle is an oval-shaped, travel-sized bottle.

7. The kit of claim 2 wherein said two-part fastener is a hook-and-loop fastener having a hook portion and a loop portion, said hook portion being permanently affixed to an opposing end of said connector strip from said slotted hole, and wherein said bottle is oval-shaped and sized to contain approximately ¾ oz. of lubricant.

8. The kit of claim 7 further comprising a plurality of cleansing towelettes.

9. The kit of claim 7 further comprising instructions for use.

10. The kit of claim 7 further comprising a pouch to carry said kit elements.

11. The kit of claim 7 further comprising a rubber band.

12. The kit of claim 7 further comprising a sealable plastic bag and an application brush.

13. A method for lubricating a catheter using a kit including a connector strip and a two-part fastener, wherein a first portion of the two-part fastener has been permanently affixed to said connector strip and a second portion of said two-part fastener has been permanently affixed to a mounting location, comprising the steps of: linking said catheter to said connector strip; temporarily attaching said connector strip to a mounting location by mating said first portion of said two-part fastener with said second portion; lubricating said catheter; inserting, using, and removing said catheter; and cleaning up.

14. The method of claim 13 wherein said connector strip defines a slotted hole and said catheter is linked to said connector strip by threading the catheter through said slotted hole in said connector strip.

15. The method of claim 13 wherein said two-part fastener is a hook-and-loop fastener having a hook portion and a loop portion, and said hook portion is permanently affixed to said connector strip and said loop portion is permanently affixed to a mounting location.

16. The method of claim 13 wherein said mounting location is located on the user's clothing.

17. The method of claim 14 wherein said user's clothing comprises a pair of pants and said mounting location is located on a rear part of the inside waistband of said pants.

18. The method of claim 16 wherein said user's clothing comprises a pair of pants and said mounting location is located on a front part of the inside waistband of said pants.

19. A method for lubricating a catheter using a kit including a connector strip, comprising the steps of: linking said catheter to said connector strip; temporarily mounting said connector strip to a mounting location; lubricating said catheter; inserting, using, and removing said catheter; and cleaning up.

20. The method of claim 19 wherein said kit also comprises a rubber band and a small bottle of lubricant, and said steps further comprise the user strapping said bottle of lubricant to his hand using said rubber band to aid in the lubrication step.
